(19) United States
(12) Patent Application Publication
(10) Pub. No.: US 2004/0223926 A1
(43) Pub. Date: Nov. 11, 2004

(54) Coating solution for forming wettability-varied pattern and method of producing pattern-formed body

(76) Inventor: Hironori Kobayashi, Tokyo-to (JP)

Correspondence Address: Richard J. Streit, Ladas & Parry, Suite 1200, 224 South Michigan Avenue, Chicago, IL, 60604, US

(21) Appl. No.: 10/786,790

(22) Filed: Feb. 25, 2004

(30) Foreign Application Priority Data
Feb. 28, 2003 (JP) 2003-053158

(51) Int. Cl. A61K 7/021

(52) U.S. Cl. 424/63

(57) ABSTRACT

The main object of the present invention is to provide a coating solution for forming a wettability-varied pattern, which solution does not allow metals to be dissolved thereinto and thus can be used for producing a pattern-formed body, and a method of producing a pattern-formed body by using the coating solution for forming a wettability-varied pattern. In order to achieve this object, the present invention provides a coating solution for forming a wettability-varied pattern, wherein the coating solution has pH in a neutral region and contains titanium oxide and polysiloxane having a substituent group which is liquid-repellant and directly bonded to each Si atom constituting polysiloxane.

### BACKGROUND OF THE INVENTION

1. Field of the Invention

The present invention relates to a coating solution for forming a wettability-varied pattern whose pH value is in a neutral range and which can be used for pattern formation of a color filter or the like, and a method of producing a pattern-formed body by using the coating solution for forming a wettability-varied pattern.

2. Description of the Related Art

Conventionally, there has been studied by the researchers including the inventors of the present invention a method of producing a pattern-formed body, which method including: forming a layer on a base material by using a coating solution for forming a wettability-varied pattern, which coating solution contains a photocatalyst and a material whose characteristic changes by the action of the photocatalyst upon irradiation of energy; and exposing the layer in a patterned-configuration, thereby forming a pattern whose wettability has been changed (Japanese Patent Application Laid-Open No. 11-344804). In this method, in order to keep the photocatalyst and the material whose characteristic changes by the action of the photocatalyst upon irradiation of energy, in a finely dispersed state, the coating solution for forming a wettability-varied pattern is used in a state in which the pH value thereof is in an acidic range.

However, devices used for producing a pattern-formed body by using a coating solution for pattern formation are generally made of metal, in order to ensure high precision in processing or the like. Thus, in such a conventional method as described above, there is a possibility that such metal or the like is dissolved into the coating solution for forming a wettability-varied pattern. In this case, there arise problems in that control on sensitivity of the photocatalyst upon irradiation of energy becomes difficult and that the coating solution for forming a wettability-varied pattern is easily gelated, due to the action of the dissolved metals or the like. Further, as the material whose characteristic changes by the action of the photocatalyst upon irradiation of energy is acidic, there arises another problem in that metal portions of the devices are easily rusted and waste liquid or the like must be treated in a satisfactory manner.

Yet further, when a material which is easily corroded by acid, such as aluminum, is used as a base material of a pattern-formed body, the base material is corroded by the coating solution for a pattern-formed body. Accordingly, it is very difficult to produce a pattern-formed body by the aforementioned conventional method.

### SUMMARY OF THE INVENTION

In view of the above-described circumstances, the present invention provides a coating solution for forming a wettability-varied pattern, which solution allows relatively little amount of metals or the like to be dissolved thereinto and thus can be used for producing a pattern-formed body, and a method of producing a pattern-formed body by using the coating solution for forming a wettability-varied pattern.

The present invention provides a coating solution for forming a wettability-varied pattern, wherein the coating solution has pH in a neutral region and contains titanium oxide and polysiloxane having a substituent group which is liquid-repellant and directly bonded to each Si atom constituting polysiloxane.

According to the present invention, the coating solution for forming a wettability-varied pattern contains titanium oxide and the above-described polysiloxane. Thus, by applying, by coating, this coating solution for forming a wettability-varied pattern, a wettability-variable layer can be obtained whose contact angle with liquid is easily decreased by the action of titanium oxide upon irradiation of energy. Further, as the pH value of the coating solution for forming a wettability-varied pattern is in a neutral range, it is possible to prevent, when the wettability-variable layer is formed by using the coating solution for forming a wettability-varied pattern, metals or the like from being dissolved into the coating solution for forming a wettability-varied pattern. As a result, the wettability of the wettability-variable layer can be changed in a stable and reliable manner by the action of titanium oxide upon irradiation of energy, whereby a pattern-formed body having a highly precise pattern formed thereon can be produced. Further, the coating solution for forming a wettability-varied pattern can be applied to a base material which is easily corroded by acid, whereby pattern-formed bodies of various types can be produced.

In the present invention, the coating solution for forming a wettability-varied pattern preferably contains alkylsilicate. When the coating solution contains alkylsilicate, titanium oxide is stably kept in a finely dispersed state with a pH value thereof in a neutral range.

In the present invention, the substituent group which is liquid-repellant is preferably a fluoroalkyl group. When the substituent having liquid-repellant properties is a fluoroalkyl group, a highly liquid-repellant layer can be obtained by applying, by coating, the coating solution for forming a wettability-varied pattern and thus a difference in wettability between a region which has been made lyophilic by irradiation of energy and a region which has not can be increased.

In the present invention, it is preferable that the aforementioned polysiloxane is a polysiloxane as a hydrolysis condensate or a co-hydrolysis condensate of a silicon compound, which silicon compound includes a silicon compound represented by YₙSiX₍₄₋ₙ₎ (Y represents an alkyl, fluoroalkyl, vinyl, amino, phenyl or epoxy group, X represents an alkoxyl group or a halogen, and n is an integer in a range of 0 to 3). Use of such polysiloxane is preferable because then a change in wettability as described above is intensified.

The invention provides a method of producing a coating solution for forming a wettability-varied pattern, comprising mixing a neutral sol solution of titanium oxide, whose pH is in a neutral range and which contains titanium oxide and alkyl silicate, with a solution of hydrolyzed fluoroalkylsilane, thereby preparing a coating solution for forming a wettability-varied pattern, wherein pH of the solution of hydrolyzed fluoroalkylsilane is adjusted in advance such that pH of the prepared coating solution for forming a wettability-varied pattern is in a range of 5 to 9.

According to the present invention, as pH of the prepared coating solution for forming a wettability-varied pattern is in the above-mentioned range, metals, metal oxides and the like are prevented from being dissolved from devices used for coating the coating solution for forming a wettability-varied pattern, whereby a wettability-varied pattern can be formed in a stable manner. Further, by adjusting in advance pH of the solution of hydrolyzed fluoroalkylsilane and then mixing the hydrolyzed solution with the above-described titanium oxide sol, a coating solution for forming a wettability-varied pattern whose pH is in a neutral range can be produced without destroying the state in which titanium oxide has been finely dispersed.

The present invention provides a method of producing a pattern-formed body, comprising the processes of: forming a wettability-variable layer whose wettability at a portion irradiated with energy is modified such that a contact angle with liquid at the portion is decreased, by coating a base material with a coating solution for forming a wettability-varied pattern whose pH is in a neutral region and which contains titanium oxide and polysiloxane having a substituent group which is liquid-repellant directly bonded to each Si atom constituting the polysiloxane and then drying or hardening the coating; and forming, on the wettability-variable layer, a wettability-varied pattern which is constituted of a lyophilic region and a liquid-repellant region, by irradiating the wettability-variable layer with energy in a pattern-like configuration.

According to the present invention, the wettability-variable layer is formed by using the coating solution for forming a wettability-varied pattern. Thus, the wettability of a surface of the layer can be easily changed by the action of titaium oxide upon irradiation of energy. By utilizing difference in wettability generated in such a manner, a variety of functional portions can be formed on the pattern-formed body.

Further, as pH of the coating solution for forming a wettability-varied pattern is in a neutral range, metals or the like of a nozzle and the like, which are used for applying by coating the coating the coating solution for forming a wettability-varied pattern, are less likely to be dissolved during the wettability-variable layer forming process. Accordingly, it is possible to effect a change in wettability upon irradiation of energy in a stable and reliable manner in the wettability-varied pattern forming process. Yet further, as the wettability-variable layer includes no acid, there is substantially no possibility that a device for exposure and the like are rusted in the wettability-varied pattern forming process, whereby a pattern-formed body can be produced in a stable manner. Yet further, as the coating solution for forming a wettability-varied pattern of the invention allows use of a base material which is easily corroded by acid, pattern-formed bodies of various types can be produced.

In the present invention, it is preferable that the method of producing a pattern-formed body further comprises the process of preparing a coating solution for forming a wettability-varied pattern, in which process a sol solution of titanium oxide containing the titanium oxide and alkylsilicate is mixed with a solution of hydrolyzed polysiloxane, prior to the wettability-variable layer forming process. Preparing the coating solution for forming a wettability-varied pattern prior to the wettability-variable layer forming process is preferable because then a wettability-variable layer can be formed in a stable manner.

In the present invention, the method may comprise the process of filtering the coating solution for forming a wettability-varied pattern, in which process the coating solution for forming a wettability-varied pattern is filtered prior to the wettability-variable layer forming process. Inclusion of the aforementioned process of filtering the coating solution for forming a wettability-varied pattern results in formation of more even wettability-variable layer. Further, according to the present invention, as pH of the coating solution for forming a wettability-varied pattern is in a neutral range, even when a filter made of stainless steel or the like is used, metals or the like are prevented from being dissolved into the coating solution for forming a wettability-varied pattern, whereby change in quality of the filtered solution, or the like can be prevented in a reliable manner.

In the present invention, coating of the coating solution for forming a wettability-varied pattern during the wettability-variable layer forming process is carried out by a method selected from the group consisting of spin coating, slit coating, bead coating, spray coating, dip coating, and combination of slit coating and spin coating. According to the present invention, as pH of the coating solution for forming a wettability-varied pattern is in a neutral range, metals or the like are prevented from being dissolved from the above-described devices into the coating solution, whereby a wettability-variable can be formed in a stable manner.

In the present invention, the coating solution for forming a wettability-varied pattern is dried or hardened in the wettability-variable layer forming process by drying the coating with a hot plate, an IR heater or an oven is preferable. According to the present invention, as pH of the coating solution for forming a wettability-varied pattern is in a neutral range, the hot plate, IR heater, oven or the like are not rusted and thus a pattern-formed body can be produced in a stable manner.

In the present invention, irradiation of energy in the wettability-varied pattern forming process may be effected by way of a mask. Further, in the present invention, a light-shielding portion may be formed on the base material so that energy irradiation in the wettability-varied pattern forming process is carried out from the base material side. Yet further, in the present invention, energy irradiation in the wettability-varied pattern forming process may be effected with laser. According to the present invention, as pH of the coating solution for forming a wettability-varied pattern is in a neutral range, the mask and the energy irradiating device are not rusted and thus a pattern resulted from change in characteristics of the wettability-variable layer can be formed on a body on which a pattern is to be formed body, in a stable manner.

The present invention provides a method of producing a functional element, comprising the process of forming a functional portion, in which process a functional portion is formed on the wettability-varied pattern of the pattern-formed body produced by the method of producing a pattern-formed body of the present invention. According to the present invention, as pH of the wettability-variable layer of the pattern-formed body is in a neutral range, the functional portion formed on the wettability-varied pattern is free of any influence of acid, whereby a functional element which is stable for a sufficiently long period can be produced. Further, as the functional portion is formed by utilizing difference in wettability between the wettability-varied pattern and other portions, it is possible to easily form a highly precise functional portion.

In the present invention, the functional portion forming process is preferably carried out by coating or discharge from a nozzle. Such methods are preferable because then a highly precise functional portion can be formed.

The present invention provides a color filter, comprising a pixel portion which is the functional portion of the functional element produced by the above-described method of producing a functional element of the present invention. According to the present invention, a color filter is obtained in which a highly precise pixel portion can be formed by the ink jet method or the like in accordance with the wettability-varied pattern. Further, a color filter of high quality, whose pixel portion is free of any influence of acid for a sufficiently long period, can be obtained.

The present invention provides a microlens, comprising a lens which is the functional portion of the functional element produced by the above-described method of producing a functional element of the present invention. According to the present invention, a microlens can be obtained in which a highly precise lens has been formed by utilizing the wettability-varied pattern. Further, a microlens of high quality, whose lens is free of any influence of acid for a sufficiently long period, can be obtained.

The present invention provides a conductive pattern, comprising a metal wiring which is the functional portion of the functional element produced by the above-described method of producing a functional element of the present invention. According to the present invention, a conductive pattern can be obtained in which a highly precise metal wiring has been formed by the electric field jet method or the like by utilizing the wettability-varied pattern. Further, a conductive pattern of stable and high quality can be obtained because the conductive pattern is free of any influence of acid for a sufficiently long period.

The present invention provides a base material for biochip, comprising the functional portion of the functional element produced by the above-described method of producing a functional element of the present invention, the functional portion being attachable to a biomaterial. According to the present invention, as the functional portion is formed by utilizing the wettability-varied pattern, a base material for a biochip can be obtained which is processable in a highly precise manner and free of any influence of acid for a sufficiently long period.

The present invention provides an organic electroluminescent (EL) element, comprising an organic EL layer which is the functional portion of the functional element produced by the above-described method of producing a functional element of the present invention. According to the present invention, an organic EL element can be obtained in which a highly precise organic EL layer is formed by utilizing the wettability-varied pattern. Further, an organic EL element of high quality, which is free of any influence of acid for a sufficiently long period, can be obtained.

The present invention provides a coating device using mixture of two types of liquids, the device being used for the method of producing a pattern-formed body, comprising: a neutral titanium oxide sol solution accommodating section for accommodating a neutral sol solution of titanium oxide; a hydrolyzed solution accommodating section for accommodating a solution of hydrolyzed fluoroalkylsilane; a stirring section connected to the neutral titanium oxide sol solution accommodating section and the hydrolyzed solution accommodating section such that the neutral sol solution of titanium oxide and the solution of hydrolyzed fluoroalkylsilane can be supplied to the stirring section and stirred therein; and a coating section for coating the base material with a coating solution for forming a wettability-varied pattern, which is prepared by the stirring of the two solutions at the stirring section. According to the present invention, the coating solution for forming a wettability-varied pattern can be prepared by mixing the two solutions immediately before the coating process and then coated on the base material. Thus, coating of the coating solution can be carried out in a stable manner without causing a problem such as deterioration of quality of the coating solution.

### BRIEF DESCRIPTION OF THE DRAWING

FIG. 1 is a process view showing one example of a method of producing a pattern-formed body of the present invention.

### DESCRIPTION OF THE PREFERRED EMBODIMENT

The present invention relates to a coating solution for forming a wettability-varied pattern, a method of producing a pattern-formed body by using the coating solution for forming a wettability-varied pattern and a method of producing a functional element using the pattern-formed body. Each of the coating solution, the method of producing a pattern-formed body and the method of producing a functional element will be described one by one, hereinafter.

### A. A Coating Solution for Forming a Wettability-Varied Pattern

First, a coating solution for forming a wettability-varied pattern of the present invention will be described. The coating solution for forming a wettability-varied pattern of the present invention is such that it has pH in a neutral region and contains titanium oxide and polysiloxane having a substituent group which is liquid-repellant and directly bonded to each Si atom constituting polysiloxane.

According to the present invention, as the coating solution for forming a wettability-varied pattern contains the above-described polysiloxane, a wettability-variable layer whose wettability at a layer surface is changed by the action of titanium oxide upon irradiation of energy can be obtained by applying, by coating, the coating solution for forming a wettability-varied pattern.

Generally, in a case in which metals or metal oxides are mixed into the wettability-variable layer, sensitivity of titanium oxide upon irradiation of energy is changed due to these metals or metal oxides at the time when wettability of the wettability-variable layer is to be modified. There is a possibility, depending on the type of metals, that the wettability is no longer modified in a stable manner, which makes the control of the wettability difficult.

In the present invention, as pH of the coating solution for forming a wettability-varied pattern is in a neutral range, dissolution of metals or the like from a device (specifically, a nozzle or the like) used for forming the wettability-variable layer, which dissolution might occur if an acidic coating solution for forming a wettability-varied pattern were to be used, can reliably be prevented. Thus, when a pattern-formed body is formed by using the coating solution for forming a wettability-varied pattern of the present invention, change in wettability can be effected stably and evenly. Further, as the coating solution for forming a wettability-varied pattern can be applied to a base material which is easily corroded with acid, pattern-formed bodies of various types can be produced.

The coating solution for forming a wettability-varied pattern of the present invention preferably contains alkylsilicate. When the coating solution contains alkylsilicate, titanium oxide can be maintained in a finely dispersed state with pH of the coating solution being in a neutral range.

Hereinafter, each of the components of the coating solution for forming a wettability-varied pattern of the present invention will be described in detail.

1. Titanium Oxide

First, titanium oxide used for the coating solution for forming a wettability-varied pattern of the present invention will be described. Titanium oxide used for the coating solution for forming a wettability-varied pattern of the present invention acts as a photocatalyst and makes the wettability at a layer surface change, at the time when the coating solution for forming a wettability-varied pattern is applied by coating on a base material. As titanium oxide (TiO₂) has high band-gap energy, is chemically stable, not toxic and is easily available, use of titanium oxide as a photocatalyst enables stable and effective application of the coating solution for forming a wettability-varied pattern of the present invention. There are two types of titanium oxide, i.e., anatase-type and rutile type. Either of these two types can be used in the present invention. Anatase-type titanium oxide is especially preferable. Excitation wavelength of anatase-type titanium oxide is no longer than 380 nm.

The smaller the grain diameter of titanium oxide is, the more effectively the photocatalytic reaction proceeds. In the present invention, the average grain diameter of titanium oxide in use is preferably no larger than 50 nm, and more preferably no larger than 20 nm.

2. Alkylsilicate

Next, alkylsilicate used in the present invention will be described. Alkylsilicate in the present invention is used as a dispersion-stabilizer for the aforementioned titanium oxide, so that the coating solution for forming a wettability-varied pattern is stably maintained in a neutral pH range.

The aforementioned alkyl silicate is a compound represented by general formula of SiₙOₙ₋₁ (OR)₂ₙ₊₂ (in the formula, Si represents silicon, O represents oxygen and R represents an alkyl group). Among the alkylsilicate represented by the above-described general formula, those in which n is in a range of 1 to 6 and R is an alkyl group having 1 to 4 carbon atoms are preferable in terms of having a relatively high proportion of silicon.

Regarding the content of the alkylsilicate, it is preferable that the weight ratio of the weight amount of silicon in the alkylsilicate, which weight amount has been converted to the weight amount of SiO₂, to the weight amount of titanium in the titanium oxide, which weight amount has been converted to the weight amount of TiO₂, i.e., SiO₂/TiO₂, is in a range of 0.7 to 10. It is more preferable that the above-described weight ratio is in a range of 0.9 to 2. When the content of alkylsilicate which has been blended is less than the aforementioned range, the dispersion stability may be deteriorated. On the contrary, when the content of alkylsilicate which has been blended is more than the aforementioned range, the photocatalytic function of titanium oxide may be deteriorated. That is, both of such extremes are not preferable.

3. Polysiloxane

Next, polysiloxane used for the coating solution for forming a wettability-varied pattern of the present invention will be described. In the polysiloxane used in the present invention, a substituent group having liquid-repellant properties is directly connected to each Si atom constituting polysiloxane. Examples of the substituent group having liquid-repellant properties include alkyl group, fluoroalkyl group, vinyl group, amino group, phenyl group and epoxy group. As the substituent group having liquid-repellant properties is directly connected to each Si atom constituting polysiloxane, good liquid-repellant properties can be exhibited at the time when the coating solution for forming a wettability-varied pattern is coated on a base material and a wettability-variable layer is formed thereon. Upon irradiation of energy on the wettability-variable layer, the substituent group is discomposed by the action of the photocatalyst and the energy-irradiated portion exhibits lyophilicity. In the present specification, the expression that “the substituent group having liquid-repellant properties is directly connected to each Si atom constituting polysiloxane” means that such a substituent as described above is directly bonded to each skeleton Si atom constituting polysiloxane, without having O atom or the like therebetween. The polysiloxane used in the present invention generally has plural alkoxy groups, acetyl groups or halogens as the substituent groups.

The type of the polysiloxane having such liquid-repellant substituent groups is not particularly limited as long as the bonding energy of the main skeleton is high enough to prevent the skeleton from being discomposed due to light-excitation of titanium oxide, at the time when polysiloxane is condensed as a result of hydrolysis and a wettability-variable layer has been formed. Examples of such polysiloxane include those which are hydrolyzed and polycondensed as a result of a sol-gel reaction or the like, to exhibit a large strength.

In this case, it is preferable that the aforementioned polysiloxane is a hydrolysis condensate or a co-hydrolysis condensate of a silicon compound, which silicon compound includes a silicon compound represented by:

YₙSiX₍₄₋ₙ₎

(Y represents an alkyl, fluoroalkyl, vinyl, amino, phenyl or epoxy group, X represents an alkoxyl group, an acetyl group or a halogen, and n is an integer in a range of 0 to 3). The aforementioned polysiloxane may be a hydrolysis condensate, a co-hydrolysis condensate, or the like, of silicon compounds of two or more types selected from the above-described examples. The number of carbon atoms of the group represented by Y is preferably in a range of 1 to 20. The alkoxy group represented by X is preferably a methoxy, ethoxy, propoxy or butoxy group.

In the present invention, a fluoroalkyl group is especially preferable among the substituent groups having liquid-repellant properties.

Specific examples of the silicon compound having a fluoroalkyl group include following fluoroalkylsilanes. Compounds known as fluorine-based silane coupling agents can generally be used.

CF₃(CF₂)₃CH₂CH₂Si(OCH₃)₃;

CF₃(CF₂)₅CH₂CH₂Si(OCH₃)₃;

CF₃(CF₂)₇CH₂CH₂Si(OCH₃)₃;

CF₃(CF₂)₉CH₂CH₂Si(OHC₃)₃;

(CF₃)₂CF(CF₂)₄CH₂CH₂Si(OCH₃)₃;

(CF₃)₂CF(CF₂)₆CH₂CH₂Si(OCH₃)₃;

(CF₃)₂CF(CF₂)₈CH₂CH₂Si(OCH₃)₃;

CF₃(C₆H₄)C₂H₄Si(OHC₃)₃;

CF₃(CF₂)₃(C₆H₄)C₂H₄Si(OHC₃)₃;

CF₃(CF₂)₅(C₆H₄)C₂H₄Si(OHC₃)₃;

CF₃(CF₂)₇(C₆H₄)C₂H₄Si(OHC₃)₃;

CF₃(CF₂)₃CH₂CH₂SiCH₃(OCH₃)₂;

CF₃(CF₂)₅CH₂CH₂SiCH₃(OCH₃)₂;

CF₃(CF₂)₇CH₂CH₂SiCH₃(OCH₃)₂;

CF₃(CF₂)₉CH₂CH₂SiCH₃(OCH₃)₂;

(CF₃)₂CF(CF₂)₄CH₂CH₂SiCH₃(OCH₃)₂;

(CF₃)₂CF(CF₂)₆CH₂CH₂SiCH₃(OCH₃)₂;

(CF₃)₂CF(CF₂)₈CH₂CH₂SiCH₃(OCH₃)₂;

CF₃(C₆H₄)C₂H₄SiCH₃(OCH₃)₂;

CF₃(CF₂)₃(C₆H₄)C₂H₄SiCH₃(OCH₃)₂;

CF₃(CF₂)₅(C₆H₄)C₂H₄SiCH₃(OCH₃)₂;

CF₃(CF₂)₇(C₆H₄)C₂H₄SiCH₃(OCH₃)₂;

CF₃(CF₂)₃CH₂CH₂Si(OCH₂CH₃)₃;

CF₃(CF₂)₅CH₂CH₂Si(OCH₂CH₃)₃;

CF₃(CF₂)₇CH₂CH₂Si(OCH₂CH₃)₃;

CF₃(CF₂)₉CH₂CH₂Si(OCH₂CH₃)₃; and

CF₃(CF₂)₇SO₂N(C₂H₅) C₂H₄CH₂Si(OCH₃)₃.

In a case in which a wettability-variable layer is formed by applying the coating solution for forming a wettability-varied pattern, the polysiloxane as a hydrolysis condensate or a co-hydrolysis condensate of a silicon compound at least containing at least one type of the above-described substances is subjected to condensation with the above-described alklysilicate, whereby a wettability-variable layer having a predetermined strength is formed.

In the present invention, in a case in which the coating solution for forming a wettability-varied pattern has a fluoroalkyl group and a wettability-variable layer is formed by applying this coating solution for forming a wettability-varied pattern, the content of fluorine at a surface of the wettability-variable layer is decreased upon irradiation of energy on the wettability-variable layer, by the action of the above-described titanium oxide, as compared with the fluorine content before the energy irradiation.

As a result, a pattern-formed body, on which a pattern constituted of portions having less fluorine content than other portions has been formed, can be easily produced by irradiating the wettability-variable layer with energy. Fluorine has extremely low surface energy and thus a portion of a material surface containing a relatively large amount of fluorine exhibits smaller critical surface tension than other portions. Accordingly, critical surface tension at a portion whose fluorine content is relatively small is larger than critical surface tension at a portion whose fluorine content is relatively large. This means that a portion whose fluorine content is relatively small is more lyophilic than a portion whose fluorine content is relatively large. Accordingly, forming a pattern constituted of portions whose fluorine content is small as compared with the surrounding surface portions means forming a pattern of a lyophilic region in a liquid-repellant region.

Thus, by using the wettability-variable layer formed as described above, a pattern-formed body can be produced on which a lyophilic region has been easily formed in a liquid-repellant region by energy irradiation of pattern-like configuration. For example, in a case in which a composition for forming a pixel portion, which composition is to form a pixel portion, is applied by ink jet coating or the like to a lyophilic region, a pattern-formed body which is applicable to a color filter having a highly precise pattern formed therein can be obtained.

4. Coating Solution for Forming a Wettability-Varied Pattern

Next, the coating solution for forming a wettability-varied pattern of the present invention will be described. The coating solution for forming a wettability-varied pattern of the present invention exhibits pH in a neutral range and contains titanium oxide and polysiloxane having a substituent group which is liquid-repellant, as described above.

In the present invention, “pH in a neural range” of the coating solution for forming a wettability-varied pattern represents pH in a range which does not cause an adverse effect (such as corrosion) to a coating device used in the processes. Specifically, it is preferable that pH of the coating solution for forming a wettability-varied pattern is in range of 5 to 9. The pH range is more preferably in a range of 6 to 8, and most preferably in a range of 6.5 to 7.5.

Because pH of the coating solution for forming a wettability-varied pattern is within the above-described range, metals and the like are reliably prevented from being dissolved into the coating solution when a wettability-variable layer is formed by using the coating solution. As a result, the wettability of the layer formed by applying, by coating, the coating solution for forming a wettability-varied pattern can be changed in a stable manner, by the action of titanium oxide upon irradiation of energy, whereby a pattern-formed body on which a highly precise pattern has been formed can be produced.

It is preferable that the neutral sol solution of titanium oxide containing titanium oxide and alkylsilicate described above and the solution of hydrolyzed polysiloxane described above are prepared separately and then mixed with each other, to produce the coating solution for forming a wettability-varied pattern of the present invention.

Such separate preparation of the neutral sol solution of titanium oxide and the solution of hydrolyzed polysiloxane is preferable because the majority of the polysiloxanes used in the present invention are not smoothly hydrolyzed in a neutral range and, due to this, these polysiloxanes are not smoothly hydrolyzed when they are added to the neutral sol solution of titanium oxide, even if heat is applied thereto.

A method of producing the coating solution for forming a wettability-varied pattern is similar to that described in following “B. A method of producing the coating solution for forming a wettability-varied pattern”. Thus, a detailed description thereof will be omitted here.

When the coating solution for forming a wettability-varied pattern of the present invention is to be produced, it is preferable that the solution of hydrolyzed fluoroalkylsilane and the neutral sol of titanium oxide are mixed with each other immediately before applying, by coating, the coating solution for forming a wettability-varied pattern. Mixing the solution of hydrolyzed fluoroalkylsilane and the neutral sol of titanium oxide in such a manner is preferable because then deterioration of the coating solution for forming a wettability-varied pattern can be prevented for a sufficiently long period and thus a body which allows stable and even formation of a pattern can be formed.

In the present specification, “mixing the solution of hydrolyzed fluoroalkylsilane and the neutral sol of titanium oxide with each other immediately before applying, by coating, the coating solution for forming a wettability-varied pattern” means that the solution of hydrolyzed fluoroalkylsilane and the neutral sol of titanium oxide are mixed with each other at a stage where the solution and the sol are charged in a device for coating of the coating solution for forming a wettability-varied pattern. Specifically, the aforementioned mixing is carried out preferably within 24 hours, more preferably within 10 hours, and most preferably within 5 hours after the preparation of the coating solution for forming a wettability-varied pattern.

As a device used for applying, by coating, the coating solution for forming a wettability-varied pattern, a coating device using mixture of two types of liquids (which may occasionally be referred to “two-liquid-mixing-and-coating device” and will be described below) may be used. Use of the two-liquid-mixing-and-coating device is preferable because then it is possible to effect coating of the coating solution for forming a wettability-varied pattern, immediately after mixing the hydrolyzed solution and the neutral sol of titanium oxide with each other.

### B. A Method of Producing the Coating Solution for Forming a Wettability-Varied Pattern

Next, a method of producing the coating solution for forming a wettability-varied pattern of the present invention will be described. The method of producing the coating solution for forming a wettability-varied pattern of the present invention comprises mixing a neutral sol solution of titanium oxide containing titanium oxide and alkylsilicate with a solution of hydrolyzed fluoroalkylsilane of which pH is within a predetermined range.

As descried above, in general, fluoroalkylsilane is hydrolyzed in an acidic condition and is not smoothly hydrolyzed in a neutral condition, even when heat is applied thereto. In the present invention, the neutral sol solution of titanium oxide containing titanium oxide and alkylsilicate and the solution of hydrolyzed fluoroalkylsilane are separately prepared and then mixed with each other. Due to this, a coating solution for forming a wettability-varied pattern, of which pH is kept stable in a neutral range, can be produced without destroying a state in which titanium oxide is finely dispersed.

The solution of hydrolyzed fluoroalkylsilane used in the present invention can be obtained by hydrolysis of the fluoroalkylsilane described with regard to the polysiloxane of “A. Coating solution for forming a wettability-varied pattern” in water, alcohol or the like containing an inorganic acid or an organic acid dissolved thereinto. In the present invention, it is preferable that the number of carbon atoms of the alcohol is no larger than 4.

The content of fluoroalkylsilane and that of alkoxysilane which is optionally used with fluoroalkylsilane, in the solution of hydrolyzed fluoroalkylsilane, are preferably 10 wt. % to 90 wt. %, and more preferably 50 wt. % to 80 wt. %, respectively.

If the solution of hydrolyzed fluoroalkylsilane is strongly acidic, such a solution may change the dispersed state of the neutral sol of titanium oxide and destroy the state in which titanium oxide is finely dispersed in the sol, when the solution is mixed with the sol. Therefore, pH of the solution of hydrolyzed fluoroalkylsilane when the solution is finally added to the neutral sol of titanium oxide is to be in a range of 2 to 7, and preferably in a range of 5 to 7.

On the other hand, the neutral sol solution of titanium oxide containing titanium oxide and alkylsilicate can be obtained by mixing alkylsilicate with titanium oxide sol and neutralizing the mixture. In the present invention, pH of the neutral sol solution of titanium oxide represents a range of pH in which titanium oxide and the like are stably dispersed in the neutral sol solution of titanium oxide. Specifically, pH of the neutral sol solution of titanium oxide is preferably in a range of 5 to 9, more preferably in a range of 6 to 8, and most preferably in a range of 6.5 to 7.5.

As the titanium oxide sol, titanium oxide sol obtained by the conventional method may be used. Examples of the conventional method include: peptizing titanium oxide such as titanium dioxide hydrate with monobasic acid or a salt thereof; adding titanium tetrachloride to cold water and then subjecting the mixture to dialysis; adding titanium alkoxide to an aqueous solution of hydrochloric acid; and the like.

Next, the thus obtained titanium oxide sol and alklysilicate are mixed with each other according to the known method. When titanium oxide sol and alklysilicate are mixed with each other, aqueous sol of titanium oxide (and optionally alkylsilicate, as well) may be diluted with a hydrophilic organic solvent. Examples of the hydrophilic organic solvent include alcohols such as methanol, ethanol, 2-propanol, ethylene glycol, ketones, esters of carboxylic acids and the like. The type of the hydrophilic organic solvent is not limited as long as the solvent is hydrophilic. Alcohol, however, is preferable because solubility of alkylsilicate thereto is high. The dilution ratio (the weight amount ratio) of the titanium oxide sol by a hydrophilic organic solvent is preferably in a range of 1.2 to 5. The dilution ratio (the weight amount ratio) of alkylsilicate by a hydrophilic organic solvent is preferably in a range of 1.5 to 5. Mixing the titanium oxide sol with alkylsilicate after these are each diluted with a hydrophilic organic solvent is preferable because then the mixing can be effected without causing titanium oxide to aggregate. The hydrophilic organic solvent for diluting the titanium oxide sol need not be the same compound as the hydrophilic organic solvent for diluting alkylsilicate.

After the aforementioned mixing, the obtained mixture is neutralized to obtain a neutral sol of titanium oxide. The neutralization of the titanium oxide sol can be carried out by a conventional method, which is preferably at least one method selected from the group consisting of an ion-exchange method, a method of adding a neutralizer and a method of utilizing dialysis. Neutralizing the titanium oxide sol by adding a neutralizer after carrying out ion exchange is preferably in terms of reducing the contents of impurities.

The ion-exchange method is carried out by using an ion-exchange resin. For example, this method includes: the processes of adding a cation-exchange resin or an anion-exchange resin to the mixture of the titanium oxide sol and alkylsilicate, thereby removing cations or anions; and separating the ion-exchange resin. The cation-exchange resin may be strongly acidic or weakly acidic. The anion-exchange resin may be either strongly basic or weakly basic. Commercially available products such as Amberlite (Organo Corporation), Diaion (Mitsubishi Chemical Co., Ltd.) and the like can be used for the ion-exchange resins.

Examples of the neutralizer which can be used include: an alkali substance such as sodium hydroxide, potassium hydroxide, and aqueous ammonia; a monobasic acid such as hydrochloric acid, nitric acid, acetic acid, chloric acid and chromic acid, and salts thereof; and acid such as sulfuric acid and hydrofluoric acid, and salts thereof.

The thus obtained neutral sol of titanium oxide may be adjusted to a desired solid-content concentration or a desired pH, in accordance with the application.

Next, by mixing the neutral sol solution of titanium oxide with the solution of hydrolyzed fluoroalkylsilane, a coating solution for forming a wettability-varied pattern whose pH is in a neutral range can be obtained. In this mixing process, the mixture ratio (the weight ratio) of the neutral sol solution of titanium oxide to the solution of hydrolyzed fluoroalkylsilane is preferably 1: 0.1 to 1 (the former number represents the weight of the neutral sol solution of titanium oxide and the latter number represents the weight of the solution of hydrolyzed fluoroalkylsilane), and more preferably 1: 0.1 to 0.5. When the mixture ratio is in the above-described range, a coating solution for forming a wettability-varied pattern whose pH is stably kept in a neutral range can be obtained.

### C. A Method of Producing a Pattern-Formed Body

Next, a method of producing a pattern-formed body of the present invention will be described. The method of producing a pattern-formed body of the present invention comprises the processes of: forming a wettability-variable layer whose wettability at a portion irradiated with energy is modified such that a contact angle with liquid at the portion is decreased, by coating a base material with a coating solution for forming a wettability-varied pattern whose pH is in a neutral region and which contains titanium oxide and polysiloxane having a substituent group which is liquid-repellant directly bonded to each Si atom constituting the polysiloxane and then drying or hardening the coating; and forming, on the wettability-variable layer, a wettability-varied pattern which is constituted of a lyophilic region and a liquid-repellant region, by irradiating the wettability-variable layer with energy in a pattern-like configuration.

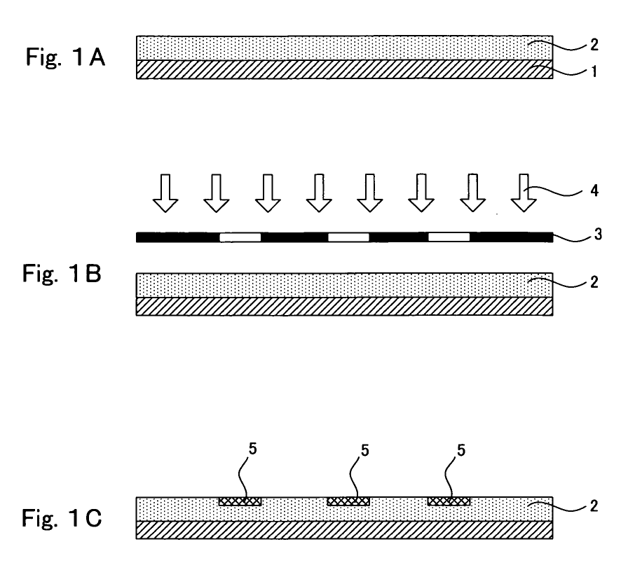

Specifically, the method of producing a pattern-formed body of the present invention is, as shown in FIG. 1, comprises the wettability-variable layer forming process (FIG. 1A) of forming a wettability-variable layer 2 by coating a base material 1 with a coating solution for forming a wettability-varied pattern whose pH is in a neutral region and which contains titanium oxide and polysiloxane including a substituent group having liquid-repellant properties and directly bonded to each Si atom constituting the polysiloxane and then drying or hardening the coating; and the wettability-varied pattern forming process for forming, on the wettability-variable layer 2, by using a photomask 3 or the like and irradiating energy 4 thereon (FIG. 1B), a wettability-varied pattern (FIG. 1C) which is constituted of a lyophilic region 5 which is a portion of the wettability-variable layer whose wettability has been modified and a liquid-repellant region which is a portion of the wettability-variable layer whose wettability has not been modified.

According to the present invention, as pH of the coating solution for forming a wettability-varied pattern is in a neutral range, dissolution of metals, metal oxides or the like from a coating device such as a nozzle, which dissolution might occur if an acidic coating solution were to be used, can reliably be prevented. As a result, a wettability-varied pattern can stably and evenly formed, without causing a problem in that control on the action of titanium oxide upon irradiation of energy becomes difficult due to metals or the like being contained in the wettability-variable layer.

Further, as no acid is contained in the wettability-variable layer, there is no possibility that acid in the wettability-variable layer is released therefrom upon irradiation of energy and causes a device for exposure to be rusted. Thus, a pattern-formed body can be produced in a stable manner. Yet further, as no acid is contained in the wettability-variable layer, the layer can be formed on a base material which is easily corroded by acid, whereby various types of pattern-formed bodies can be produced.

The method of producing a pattern-formed body of the present invention may further include the process of preparing a coating solution for forming a wettability-varied pattern, in which process a coating solution for forming a wettability-varied pattern is prepared prior to the wettability-variable layer forming process, or the process of filtering the coating solution for forming a wettability-varied pattern, in which process the coating solution for forming a wettability-varied pattern is filtered prior to the wettability-variable layer forming process.

Hereinafter, each of the processes of the method of producing a pattern-formed body of the present invention will be described.

1. Wettability-Variable Layer Forming Process

First, the wettability-variable layer forming process in the method of producing a pattern-formed body of the present invention will be described. Specifically, the wettability-variable layer forming process in the method of producing a pattern-formed body of the present invention is a process of forming a wettability-variable layer whose wettability at a portion irradiated with energy is modified such that a contact angle with liquid at the portion is decreased, by coating a base material with a coating solution for forming a wettability-varied pattern whose pH is in a neutral region and which contains titanium oxide and polysiloxane including a substituent group having liquid-repellant properties and directly bonded to each Si atom constituting the polysiloxane; and then drying or hardening the coating. Hereinafter, each of the components required for the present process will be described.

(The Coating Solution for Forming a Wettability-Varied Pattern)

First, the coating solution for forming a wettability-varied pattern used in the present process will be described. The coating solution for forming a wettability-varied pattern used in the present process has pH in a neutral region and contains titanium oxide and polysiloxane having a substituent group which is liquid-repellant directly bonded to each Si atom constituting the polysiloxane. In the wettability-variable layer formed by applying, by coating, the coating solution for forming a wettability-varied pattern, wettability at a portion thereof irradiated with energy is modified by the action of titanium oxide such that a contact angle with liquid at the portion is decreased. Due to this, it is possible to form, on a body on which a pattern is to be formed, a wettability-varied pattern which is constituted of a lyophilic region as a portion of the wettability-variable layer which has been irradiated with energy and a liquid-repellant region as a portion of the wettability-variable layer which has not been irradiated with energy.

In the present specification, a “lyophilic region” represents a region where a contact angle with liquid is relatively small. For example, a lyophilic region represents a region which exhibits excellent wettability to a composition for forming a functional portion which forms a functional portion. In contrast, a “liquid-repellant region” represents a region where a contact angle with liquid is relatively large. For example, a liquid-repellant region represents a region which exhibits poor wettability to a composition for forming a functional portion which forms a functional portion.

In the present invention, one portion (region) of the wettability-variable layer is regarded as a lyophilic region when the contact angle thereat with a liquid is at least 1° smaller than the contact angle with the liquid at another portion (region) of the wettability-variable layer adjacent at the one portion. In contrast, one portion (region) of the wettability-variable layer is regarded as a liquid-repellant region when the contact angle thereat with a liquid is at least 1° larger than the contact angle with the liquid at another portion (region) of the wettability-variable layer adjacent at the one portion.

In the present invention, difference in contact angle with respect to a composition for forming a functional portion, which composition forms a functional portion, between at a lyophilic region formed by energy irradiation and at a liquid-repellant region which has not been irradiated with energy is preferably no smaller than 1°, more preferably no smaller than 5°, and most preferably no smaller than 10°.

It is preferable that a portion which has not been irradiated with energy, of the wettability-variable layer formed by applying, by coating, the coating solution for forming a wettability-varied pattern, i.e., a liquid-repellant region, has wettability in which a contact angle with a liquid having surface tension of 40 mN/m is no smaller than 10°, more preferably a contact angle thereat with a liquid having surface tension of 30 mN/m is no smaller than 10°, and most preferably a contact angle thereat with a liquid having surface tension of 20 mN/m is no smaller than 10°. A portion which has not been irradiated with energy is a portion which must exhibit liquid-repellant properties. When a contact angle with liquid at the non-irradiated portion is too small, the portion cannot exhibit satisfactory liquid-repellant properties and there is a possibility that the composition for forming a functional portion remains thereon at the time of forming a functional portion on a pattern-formed body, which is not preferable.

It is preferable that a contact angle with liquid at a portion which has been irradiated with energy, of the wettability-variable layer formed by applying, by coating, the coating solution for forming a wettability-varied pattern, is decreased such that a contact angle thereat with a liquid having surface tension of 40 mN/m is no larger than 9°, more preferably a contact angle thereat with a liquid having surface tension of 50 mN/m is no larger than 10°, and most preferably a contact angle thereat with a liquid having surface tension of 60 mN/m is no larger than 10°. If a contact angle with liquid at a portion which has been irradiated with energy, i.e., a lyophilic region, is too high, there is a possibility that the composition for forming a functional portion poorly spreads at the portion at the time of forming a functional portion on a pattern-formed body. In other words, there is a possibility that deficiency in the functional portion or the like may occur.

In the present specification, “a contact angel with a liquid” is obtained from the results or a graph plotted on the basis of the results of the measurement of contact angles with respect to liquids having different surface tensions by using a contact angle-measuring device (CA-Z type manufactured by Kyowa Chemical Industry Co., Ltd.) (each contact angle is measured 30 seconds after putting a drop of each liquid by way of a microsyringe). In the aforementioned measurement, wetting index standard solutions manufactured by JUNSEI CHEMICAL CO., LTD. are used as the liquids having different surface tensions.

It is preferable that the wettability-variable layer formed by applying, by coating, the coating solution for forming a wettability-varied pattern preferably contains fluorine. With regard to the content of fluorine contained in the wettability-variable layer, the fluorine content in a lyophilic region which has been formed by energy irradiation thereon and exhibits a low fluorine content is to be no larger than 10, preferably no larger than 5, and most preferably no larger than 1, as compared with the fluorine content in a region which has not been irradiated with energy, expressed as 100.

By setting the content of fluorine contained in the wettability-variable layer, there can be generated a sufficiently large difference in lyophilicity between an energy-irradiated portion and a non-irradiated portion. Accordingly, by forming a wettability-varied pattern on the wettability-variable layer as described above, a wettability-varied pattern can be accurately formed only in a lyophilic region having a decreased fluorine content and a pattern-formed body, on which a pattern has been formed in a highly precise manner, can be obtained. It should be noted that, in the present invention, the rate of decrease in the fluorine content is calculated based on a decrease in weight.

Measurement of the fluorine content in the wettability-variable layer as described above can be carried out by various methods which are commonly practiced. Examples of such a method include X-ray photoelectron spectroscopy, ESCA (Electron Spectroscopy for Chemical Analysis), fluorescent X-ray analysis, mass spectroscopy and the like. The method is not limited to these examples and may be any method, as long as the method enables quantitative measurement of fluorine content at a surface.

In the present invention, titanium oxide is used as a photocatalyst, as described above. In a case in which titanium oxide is used as a photocatalyst as in the present invention, with regard to the fluorine content in the wettability-variable layer, which fluorine content is analyzed by X-ray photoelectron spectroscopy and quantified, it is desirable that fluorine (F) is contained in the wettability-variable layer surface at an index of no smaller than 500, preferably no smaller than 800, and more preferably no smaller than 1200, with respect to the index of titanium (Ti) as an element, which is expressed as 100.

By setting the fluorine (F) content in the wettability-variable layer in the above-described range, critical surface tension at the wettability-variable layer surface can be suppressed sufficiently low and liquid-repellant properties thereat can be reliably obtained, whereby difference in wettability between such a liquid-repellant region and a lyophilic region as a surface of a patterned portion whose fluorine content has been decreased by pattern-like irradiation of energy can be increased and precision achieved in the pattern-formed body as the final product can be significantly enhanced.

In such a pattern-formed body produced as described above, with regard to the fluorine content in the lyophilic region formed by patterned irradiation of energy, it is desirable that fluorine (F) as an element is contained in the region at an index of no larger than 50, preferably no larger than 20, and most preferably no larger than 10, with respect to the index of titanium (Ti) as an element, expressed as 100.

In a case in which the content of fluorine in the lyophilic portion of the wettability-variable layer can be decreased to such an extent as described above, the lyophilicity of the lyophilic portion is made high enough for allowing formation of a functional portion thereon. As a result, due to a sufficient difference in wettability between the lyophilic portion and the liquid-repellant portion which has not been irradiated with energy, a wettability-varied pattern can be formed on the lyophilic portion in a highly precise manner, whereby a pattern-formed body, on which a highly useful pattern has been formed, can be obtained.

It should be noted that the coating solution for forming a wettability-varied pattern used in the present process is similar to that described in the aforementioned “A. A coating solution for forming a wettability-varied pattern”. Thus, a detailed description thereof will be omitted here.

(Base Material)

Next, the base material used in the present process will be described. The type of the base material used in the present process is not particularly limited, as long as the wettability-variable layer can be formed on the base material by applying thereto, by coating, the above-described coating solution for forming a wettability-varied pattern. The type of the base material may be appropriately selected in accordance with the object for which a pattern-formed body is used and the method of energy irradiation described below. For example, in a case in which energy irradiation described below is carried out from the base material side, the base material needs to be transparent.

The base material used in the present invention may be either those which are flexible such as a film made of resin or those which are inflexible such as a glass substrate.

In the present invention, a coating solution for forming a wettability-varied pattern having pH in a neutral range is used as described above. Thus, even in a case in which a material which is easily corroded by acid, such as aluminum, is used as the base material, a pattern-formed body can be produced in a stable manner without a possibility that the base material is corroded. Therefore, use of a material which is easily corroded by acid as the base material is preferable in terms of making the best use of the advantage of the present invention.

A primer layer may be formed on the base material, in order to improve the adhesion between the base material surface and the wettability-variable layer. Examples of such a primer layer include those of silane-based or titanium-based coupling agent.

In the present invention, a member which is required for a functional element, such as a light-shielding portion, an electrode layer or the like, maybe provided on the base material.

In a case in which a light-shielding portion is formed on the base material, it is possible, in the wettability-varied pattern forming process described below, to modify wettability of the wettability-variable layer surface at which the light-shielding portion is not provided by effecting irradiation from the base material side, without carrying out drawing by way of a mask and laser. Accordingly, alignment of a substrate as a body on which a pattern is to be formed, with a mask, is renderedunnecessary, whereby the whole process can be made simple. Further, a costly device required for irradiation for drawing is also rendered unnecessary, which is advantageous in terms of cost reduction.

With regard to the position at which the light-shielding portion as described above is formed, the light-shielding portion may be formed directly on the base material, with the wettability-variable layer formed on the light-shielding portion. That is, the light-shielding portion may be provided between the base material and the wettability-variable layer. Alternatively, the light-shielding portion may be formed, in a pattern-like configuration, on a surface of the base material on which surface the wettability-variable layer is not formed.

A method of forming the light-shielding portion as described above is not particularly restricted, and any appropriatemethodmaybeselectedforapplication, inaccordance with the characteristics of a surface on which the light-shielding portion is formed, the light-shielding properties required with respect to the energy in use, and the like.

The light-shielding portion may be formed by forming a thin film of metal such as chrome, having thickness of 1000 to 2000 Å, by spattering, vacuum deposition or the like, and effecting patterning of the thin film. As a method of patterning, the conventional patterning method such as spattering may be employed.

Alternatively, the light-shielding portion may be provided by forming a layer containing resin binder and light-shielding particles such as carbon particles, metal oxides, inorganic pigments and organic pigments, in a pattern-like configuration. Examples of the resin binder which may be used include: resin such as polyimide resin, acryl resin, epoxy resin, polyacrylamide, polyvinyl alcohol, gelatin, casein, cellulose, or a mixture of these resins; photosensitive resin; a resin composition of O/W emulsion type, such as that obtained by emulsifying a reactive silicone; and the like. The thickness of the light-shielding portion made of resin as described above may be set within a range of 0.5 to 10 μm. As a method of effecting patterning of the light-shielding portion made of resin, methods such as photolithography and printing, which are conventionally employed, can be used.

(Application, by Coating, of the Coating Solution for Forming a Wettability-Varied Pattern)

In the present process, the wettability-variable layer is formed by applying, by coating, the coating solution for forming a wettability-varied pattern, to the base material. In general, metal is used in consideration of precision and easiness in processing, in a device which effects coating of the coating solution for forming a wettability-varied pattern to form a body on which a pattern is to be formed. In the present invention, as the coating solution for forming a wettability-varied pattern has pH in a neutral range, metals or the like of a device such as a nozzle for coating the coating solution for forming a wettability-varied pattern is reliably prevented from being dissolved into the coating solution for forming a wettability-varied pattern, whereby there is very little possibility that metal or the like is eventually contained in the formed wettability-variable layer. Further, the device used for the coating of the coating solution for forming a wettability-varied pattern is reliably prevented from being rusted, whereby the wettability-variable layer can be formed in a stable and reliable manner.

A method of effecting coating of the coating solution for forming a wettability-varied pattern in the present process is not particularly restricted, as long as the method enables coating of the coating solution for forming a wettability-varied pattern. In the present invention, specific examples of such a coating method include spin coating, slit coating, bead coating, spray coating, dip coating, and a combination of slit coating and spin coating. By employing these methods, not only a wettability-variable layer having a large area can be formed but also a wettability-variable layer having even thickness can be formed. In the coating method as described above, a two-liquid-mixing-and-coating device which will be described below may be used. In a case in which the coating solution for forming a wettability-varied pattern is constituted of two different liquids, use of a two-liquid-mixing-and-coating device enables mixing these two liquids immediately before coating of the coating solution for forming a wettability-varied pattern, whereby deterioration of the coating solution for forming a wettability-varied pattern due to the lapse of time can be prevented and thus the wettability-variable layer can be formed stably and evenly.

(Drying or Hardening)

Next, in the present process, after the coating solution for forming a wettability-varied pattern is applied by coating, the applied coating solution for forming a wettability-varied pattern is dried or hardened, to obtain the wettability-variable layer. In the present invention, as the coating solution for forming a wettability-varied pattern has pH in a neural range, it is possible, during this drying or hardening process, to prevent a hot plate or an IR heater from being rusted if the coating solution for forming a wettability-varied pattern attaches thereto. Further, as no acid is released from the coating solution for forming a wettability-varied pattern during the drying or hardening process, the inside of an oven is reliably prevented from being rusted by acid.

The method of drying or hardening the coating solution for forming a wettability-varied pattern is not particularly restricted. In the present invention, drying or hardening of the coating solution for forming a wettability-varied pattern is preferably carried out with a hot plate, an IR heater or an oven, because then a wettability-variable layer can be formed evenly.

2. Wettability-Varied Pattern Forming Process

Next, the wettability-varied pattern forming process in the method of producing a pattern-formed body of the present invention will be described. The pattern forming process in the method of producing a pattern-formed body of the present invention is a process of forming a wettability-varied pattern constituted of a lyophilic region and a liquid-repellant region on the wettability-variable layer by irradiating the wettability-variable layer with energy in a pattern-like configuration.

In the present process, as pH of the coating solution for forming a wettability-varied pattern is in a neutral range, metals and metal oxides are hardly contained in the wettability-variable layer. Thus, the action of titanium oxide upon irradiation of energy is demonstrated in a stable manner and thus pattern formation can be evenly carried out.

The method of energy irradiation in the present process is not particularly restricted as long as the method enables modifying wettability of the wettability-variable layer in a configuration of the desired pattern. In the present invention, as no acid is contained in the wettability-variable layer, there is no possibility that, upon energy irradiation, acid is released due to the irradiated energy and that an energy source, a mask and the like are corroded by acid.

In the present invention, the term “energy irradiation (exposure)” represents a concept which contains any type of irradiation of energy beam capable of modifying wettability of the wettability-variable layer surface. In other words, the energy irradiation of the present invention is not restricted to irradiation of visible light.

In the present process, irradiation of energy may be carried out by way of a mask such as a photomask, on which a desired pattern has been formed. Use of such a mask enables irradiation of energy in a configuration of the desired pattern, whereby wettability of the wettability-variable layer can be modified in a pattern-like configuration. The type of the mask to be used is not particularly restricted as long as the mask allows irradiation of energy in a configuration of the desired pattern. The mask may be a photomask made of a material which allows transmission of energy and includes a light-shielding portion formed thereon. Alternatively, a shadow mask in which holes are formed in a configuration of the desired pattern may be used. Specific examples of the material of the mask as described above include an inorganic material such as metal, glass and ceramic, an organic material such as plastic, and the like.

In the energy irradiation of the present invention, in a case in which a light-shielding portion is formed on the base material, by utilizing the light-shielding portion, exposure may be effected on the entire surface of the base material from the base material side. In this case, energy is irradiated only onto a portion of the wettability-variable layer at a position where the light-shielding portion has not been formed, whereby the wettability of the wettability-variable layer is modified only at the exposed portion. This specific method has an advantage in that no drawing task using a mask, laser and the like is required and thus alignment task and provision of costly drawing devices are rendered unnecessary.

The wavelength of light used for energy irradiation as described above is set generally in a range of 400 nm or shorter, preferably in a range of 380 nm or shorter. These ranges are preferable because light having wavelength in the aforementioned ranges is preferable as energy for activating the photocatalytic function of titanium oxide used as the photocatalyst.

Examples of the light source which can be used for energy irradiation as described above include mercury lamp, metal halide lamp, xenon lamp, EXCIMER lamp, and other lamps of various types.

Further, in the present invention, energy irradiation may be carried out by using laser such as EXCIMER, YAG and the like. When energy irradiation is carried out by using laser, wettability of the wettability-variable layer can be modified in a highly precise manner, without necessitating alignment of a photomask and the like and formation of a light-shielding portion on the base material.

The amount of energy to be irradiated upon the energy irradiation described above is set at the amount of irradiation necessary for effecting modification of wettability at the wettability-variable layer surface by the action of titanium oxide contained in the wettability-variable layer.

Heating the wettability-variable layer upon irradiation of energy is preferable, because then sensitivity of the wettability-variable layer is enhanced and modification of wettability can be carried out effectively. Specifically, it is preferable that the wettability-variable layer is heated to a temperature in a range of 30 to 80° C.

3. Other Processes

The method of producing a pattern-formed body of the present invention may further include, in addition to the processes described above, the process of preparing the coating solution for forming a wettability-varied pattern and the process of filtering the coating solution for forming a wettability-varied pattern. These processes will be described hereinafter.

(The Process of Preparing the Coating Solution for Forming a Wettability-Varied Pattern)

First, the process of preparing the coating solution for forming a wettability-varied pattern of the present invention will be described. In the present invention, prior to the wettability-variable layer forming process described above, the process of preparing the coating solution for forming a wettability-varied pattern may be carried out, in which process the sol solution of titanium oxide containing titanium oxide and alkylsilicate described above is mixed with the solution of hydrolyzed polysiloxane. By carrying out this process of preparing the coating solution for forming a wettability-varied pattern, it is possible to avoid deterioration of the coating solution for forming a wettability-varied pattern due to the lapse of time and form a wettability-variable layer on which a pattern can be formed evenly in a stable manner.

It is preferable that preparation of the coating solution for forming a wettability-varied pattern is carried out immediately before the coating of the coating solution for forming a wettability-varied pattern. By carrying out preparation in such a manner, deterioration of the coating solution for forming a wettability-varied pattern due the lapse of time, which makes the coating solution unstable to energy irradiation, can be prevented, whereby a pattern can be formed evenly on the wettability-variable layer in a stable manner. In the present specification, “mixing the titanium oxide sol and the solution of hydrolyzed fluoroalkylsilane is carried out immediately before coating of the coating solution for forming a wettability-varied pattern” means that the titanium oxide sol and the solution of hydrolyzed fluoroalkylsilane are mixed with each other at a stage where the sol and the solution are charged in a device used for the coating of the coating solution for forming a wettability-varied pattern.

The materials and the method of preparation employed in the present process are similar to those described in the method of producing the coating solution for forming a wettability-varied pattern. Thus, detailed descriptions thereof here will be omitted.

(The Process of Filtering the Coating Solution for Forming a Wettability-Varied Pattern)

Next, the process of filtering the coating solution for forming a wettability-varied pattern of the present invention will be descried. In the present invention, the process of filtering the coating solution for forming a wettability-varied pattern may be carried out prior to the wettability-variable layer forming process. The process of filtering the coating solution for forming a pattern of the present invention is a process of filtering the coating solution for forming a wettability-varied pattern. Due to this filtering process, impurities and the like are filtered off from the coating solution and the wettability-variable layer can be evenly formed in the wettability-variable layer forming process.

In the present invention, as pH of the coating solution for forming a wettability-varied pattern is stably maintained in a neutral range, even if a stainless-made filtering device or the like is used in the filtering process, metals and the like are prevented from being dissolved into the coating solution for forming a wettability-varied pattern. Thus, the filtered coating solution is free of problems such as gelation of the coating solution, deterioration in quality of the coating solution such as change in sensitivity to energy irradiation, and the like caused by dissolved metals or the like. Further, the filtering device itself is prevented from being rusted. As a result, a pattern-formed body can be formed in a stable manner.

In the present process, the method of filtration is not particularly restricted as long as the method enables filtration of the coating solution for forming a wettability-varied pattern. The conventional filtering method may be employed.

Examples of the filtering method used in the present process include pressure filtration using a membrane filter, and the like.

### D. Method of Producing a Functional Element

Next, the method of producing a functional element of the present invention will be described. The method of producing a functional element of the present invention is a method comprising the process of forming a functional portion, in which process a functional portion is formed on the wettability-varied pattern of the pattern-formed body produced by the method of producing a pattern-formed body of the present invention. In the pattern-formed body produced by the method of producing a pattern-formed body as described above, a wettability-varied pattern constituted of a lyophilic region and a liquid-repellant region has been formed on the surface thereof. In the present invention, by utilizing difference in wettability between the wettability-varied pattern and the surrounding region, a functional portion can be easily formed in a highly precise manner.

Further, according to the present invention, pH of the wettability-variable layer of the pattern-formed body is kept in a neutral range. Thus, in a case in which a functional portion is formed on the wettability-varied pattern, the resulting functional element is free of any influence of acid and stable for a long time.

In the present specification, the term “function” of a functional portion may represent various functions including optical functions (such as selectively-light-absorbing, light-reflecting, light-polarizing, selectively-light-transmitting, non-linear-optical functions, luminescence such as fluorescence or phosphorescence, photochromic function and the like), magnetic functions (such as hard magnetic, soft magnetic, non-magnetic, and magnetic permeable functions), electric/electronic functions (such as conductive, insulating, piezoelectric, pyroelectric, and dielectric functions), chemical functions (such as adsorptive, desorptive, catalytic, water-absorbing, ionic conductive, oxidative/reductive, elctrochemical, and electrochromic functions), mechanical functions (such as wear-resistant function), thermal function (such as heat-transferring, heat-insulating, and IR-radiating functions) and biodynamical functions (such as bio-compatible, and anti-thrombogenic functions).

The type of the composition for forming a functional portion used in the present invention varies significantly, as described above, depending on the function of the functional element, a method of forming a functional element and the like. Examples of the composition for forming a functional portion include a composition typically represented by UV-harden able monomer which has not been diluted with a solvent, a composition in a liquid state which has been diluted with a solvent, and the like. The lower the viscosity of the composition for forming a functional portion, the better, because such a composition having low viscosity enables quick formation of a pattern. In a case in which a liquid-type composition which has been diluted with a solvent is used, as increase in viscosity and change in surface tension occur due to volatilization of a solvent at the time of pattern formation, a low volatile solvent is preferably used.

The composition for forming a functional portion used in the present invention may form a functional portion either by being arranged on the lyophilic region in a state in which the composition is attached to the region or by being arranged on the lyophilic region and then subjected to treatment with a chemical, UV, heat or the like, to form a functional portion. Adding a component which is hardened by UV, heat, electron beam or the like, as a binder, to the composition for forming a functional portion is preferable because then a functional portion can be formed quickly by carrying out a hardening treatment.

In the present invention, preferable examples of the method of carrying out the functional portion forming process, in which process a functional portion is formed, include: coating such as dip coating, roll coating, blade coating and spin coating; and injection through a nozzle such as ink jet, electric field jet and a method using a dispenser. The aforementioned examples are preferable because use of these methods enables even and highly precise formation of a functional portion.

Examples of the functional element whose functional portion can be formed by the present invention include a color filter whose functional portion is a pixel portion, a microlens whose functional portion is a lens, a conductive pattern whose functional portion is metal wiring, a base material for biochip whose functional portion is attachable to a biomaterial, an organic electroluminescent element whose functional portion is an organic electroluminescent layer, and the like.

### E. Color Filter

Next, the color filter of the present invention will be described. The color filter of the present invention comprises a pixel portion which is the functional portion of the functional element produced by the above-described method of producing a functional element. According to the present invention, the pixel portion is formed by utilizing the aforementioned difference in wettability between the wettability-varied pattern and the portions surrounding the pattern, and thus the color filter is easily formed in a highly precise manner by, for example, ink jet. Further, as the aforementioned wettability-variable layer is formed with the coating solution for forming a wettability-varied pattern of which pH is in a neutral range, the eventually obtained color filter has a pixel portion of high quality, which pixel portion is free of any influence of acid or the like for a long period.

In a case in which a light-shielding portion is formed on the base material, as described above, the light-shielding portion can be used as black matrix. Accordingly, in this case, by forming a pixel portion (a colored layer) as a functional portion on the pattern-formed body of the present invention described above, a color filter can be obtained without necessitating separately forming black matrix.

### F. Microlens

Next, the microlens of the present invention will be described. The microlens of the present invention comprises a lens which is the functional portion of the functional element produced by the above-described method of producing a functional element.

The microlens of the present invention is obtained, for example, by first forming the above-described lyophilic region in a circular shape; then dropping a composition for forming a lens (the composition for forming a functional portion) on the lyophilic region, so that the composition for forming a lens spreads only on the lyophilic region whose wettability has been modified; optionally further dropping the composition for forming a lens such that a contact angle of liquid drop with respect to the lyophilic region surface is changed; and hardening the composition for forming a lens, thereby obtaining a lens having one of various shapes and/or one of various focal lengths. A highly precise microlens can be obtained in such a manner. According to the present invention, as no acid or the like is contained in the wettability-variable layer, the eventually obtained lens is free of any influence of acid or the like for a longtime. That is, a microlens of high quality can be obtained.

### G. Conductive Pattern

Next, the conductive pattern of the present invention will be described. The conductive pattern of the present invention comprises metal wiring which is the functional portion of the functional element produced by the above-described method of producing a functional element. According to the present invention, metal paste or the like is applied, by coating, along the above-described wettability-varied pattern by the electric field jet method or the like, so that a conductive pattern on which a highly precise metal wiring has been formed is obtained. According to this embodiment of the present invention, as no acid or the like is contained in the wettability-variable layer as described above, the metal wiring formed on the wettability-variable layer is free of problems such as oxidization and thus a conductive pattern of high quality can be obtained.

In the present invention, as the conductive pattern is formed on the wettability-variable layer, it is preferable to use a coating solution for forming a wettability-varied pattern which eventually forms a wettability-variable layer whose electric resistance is in a range of 1×10⁸ Ω·cm to 1×10¹⁸ Ω·cm, preferably in a range of 1×10¹² Ω·cm to 1×10¹⁸ Ω·cm. By using such a coating solution, a conductive pattern of excellent quality can be obtained.

### H. Base Material for Biochip

Next, the base material for biochip of the present invention will be described. The base material for biochip of the present invention comprises the functional portion of the functional element produced by the above-described method of producing a functional element, the functional portion being attachable to a biomaterial. In the present invention, such a base material for biochip as described above can be obtained by attaching a material attachable to a biomaterial on the lyophilic region of the aforementioned wettability-varied pattern. According to the present invention, as no acid or the like is contained in the wettability-variable layer described above, a base material for biochip of high quality can be obtained, which is prevented from being deteriorated for a long period.

By fixing a biomaterial on the base material for biochip as describe above, a biochip can be obtained. At a surface of such a biochip as this, the functional thin film functions as a fixing layer. Specifically, a biomaterial such as DNA, a protein or the like is fixed on the functional thin film, in accordance with various applications.

As the technology for fixing a biomaterial as described above, the fixing technologies keenly studied in the research and development of a bioreactor in which an enzyme is fixed on an insoluble carrier can be applied. The details of the technologies are described, for example, in “Fixed Enzymes”, Ichiro Chibatake, Kodansha Scientific, 1975, and the references listed therein.

In some biochips, electrical reading needs to be employed. In such a case, an electrode must be formed on a surface of the base material for biochip. In this case, an electrode may be formed on a surface of the base material for biochip according to the method described above with regard to the conductive pattern. Alternatively, an electrode may be formed thereon by the conventional method using photoresist.

### I. Organic Electroluminescent (EL) Element

Next, the organic EL element of the present invention will be described. The organic EL element of the present invention comprises an organic electroluminescent layer which is the functional portion of the functional element produced by the above-described method of producing a functional element. According to the present invention, as the functional portion is an organic EL layer, provision (by coating) of an organic EL layer in a patterned configuration can be easily carried out by utilizing the above-described wettability-varied pattern, whereby a highly precise organic EL element can be produced. Further, as no acid or the like is contained in the wettability-variable layer described above, an organic EL element, which is stable for a long period, can be obtained.

The organic EL element of the present invention can be obtained, for example, by forming a first electrode layer on a surface of the base material; forming the wettability-variable layer on the first electrode layer; forming a wettability-varied pattern on the wettability-variable layer, obtaining a pattern-formed body; forming an organic EL layer only at a lyophilic region by utilizing the wettability-varied pattern; and forming a second electrode layer on the organic EL layer.

J. Two-Liquid-Mixing-and-Coating Device

Next, the two-liquid-mixing-and-coating device of the present invention will be described. The two-liquid-mixing-and-coating device of the present invention is a coating device using mixture of two types of liquids, used for the above-described method of producing a pattern-formed body, comprising: a neutral titanium oxide sol solution accommodating section for accommodating the aforementioned neutral sol solution of titanium oxide; a hydrolyzed solution accommodating section for accommodating the solution of hydrolyzed fluoroalkylsilane; a stirring section connected to the neutral titanium oxide sol solution accommodating section and the hydrolyzed solution accommodating section such that the neutral sol solution of titanium oxide and the solution of hydrolyzed fluoroalkylsilane can be supplied to the stirring section and stirred therein; and a coating section for coating the base material with a coating solution for forming a wettability-varied pattern, which is prepared by the stirring of the two solutions at the stirring section.

According to the present invention, the neutral titanium oxide sol solution and the solution of hydrolyzed fluoroalkylsilane of the coating solution for forming a pattern can be accommodated separately. Further, the two solutions can be mixed and stirred immediately before the coating process. Thus, a pattern-formed body can be produced in a stable manner, without a concern of deterioration in quality of the coating solution for forming a pattern.

The neutral titanium oxide sol solution accommodating section and the hydrolyzed solution accommodating section accommodate the neutral sol solution of titanium oxide and the solution of hydrolyzed fluoroalkylsilane, respectively. The stirring section is a section in which the neutral sol solution of titanium oxide and the solution of hydrolyzed fluoroalkylsilane are blended at a predetermined blending ratio and then mixed with each other so that the resulting coating solution for forming a pattern is homogeneous. The coating section is a section in which the coating solution prepared by stirring at the stirring section is coated on a target surface. Examples of the coating section include a spin coater, a slit coater, a bead coater and the like.

The present invention is not restricted to the above-described embodiment. The above-described embodiment is provided only for illustrating the present invention, and technological scope of the present invention includes whatever has substantially the same structure and causes substantially the same effect as the technological thoughts described in the accompanying claims.

### EXAMPLES

Hereinafter, the present invention will be described further in details by the following examples.

<Method of Producing Neutral Sol of Titanium Oxide>

Acidic sol of titanium oxide “STS-01” (trade name, manufactured by Ishihara Sangyo Kaisha, Ltd.) was mixed with a dispersion stabilizer “Methyl Silicate 51” (trade name, general formula: SiₙOₙ₋₁(OCH₃)₂ₙ₊₂, n is in a range of 3 to 5, manufactured by COLCOTE CO. JP). Anion-exchange resin “Amberlite IRA-910” (trade name, manufactured by Organo Corporation), which had been moisturized, was added to the mixture with stirring. The mixture was neutralized by ion exchange. Next, the ion-exchange resin was filtered off and methanol was added, whereby a neutral sol solution of titanium oxide having pH 6.4 and the solid content of 1% was obtained. In this sample, the weight ratio of the weight amount of silicon in the methylsilicate, which weight amount was converted to the weight amount of SiO₂, to the weight amount of titanium in the titanium oxide, which weight amount was converted to the weight amount of TiO₂, i.e., SiO₂/TiO₂, was 1 (refer to JP-A 2000-53421).

<Method of Producing the Solution of Hydrolyzed Fluoroalkylsilane>

30 g of isopropyl alcohol, 3 g of fluoroalkylsilane (TSL8233, manufactured by GE Toshiba Silicones) and tetramethoxysilane (TSL8114, manufactured by GE Toshiba Silicones) and 2.5 g of 0.05N hydrochloric acid were mixed with each other and stirred for 8 hours. The mixture was diluted 100 times with isopropyl alcohol, whereby a solution of hydrolyzed fluoroalkylsilane was prepared.

<Mixture of the Neutral Titanium Oxide and the Solution of Hydrolyzed Fluoroalkylsilane>

50 g of the neutral sol of titanium oxide and 0.15 g of the solution of hydrolyzed fluoroalkylsilane were mixed with each other, whereby a composition for the wettability-variable layer, which was substantially neutral (pH 5.7) and basically ink-repellant, was obtained.

<Production of a Liquid-Repellant Layer Containing Photocatalyst>

The thus obtained composition for the wettability-variable layer was applied, by coating with a slit coater, to a glass substrate of370×470×0.7 mm, whereby a photocatalyst-containing layer of 0.15 μm thickness was obtained.

<Production of a Pattern-Formed Body>

The glass substrate having the wettability-variable layer thus formed thereon was subjected to exposure by an extra-high pressure mercury lamp (30 mW/cm², 365 nm) for 30 seconds, by way of a photomask having lines of 20 μm formed therein with 100 μm pitches between the lines, whereby a pattern-formed body having a pattern whose wettability was different from that of the surrounding regions was obtained.

<Formation of a Color Filter>

A thermosetting ink for red color filter layer (viscosity: 5 cP) was discharged to the region of the pattern-formed body where the wettability thereof had been modified, by using a piezo-driven ink jet device. The ink spread over the region where the wettability had been modified. The pattern-formed body was then subjected to a heating treatment, whereby a color filter layer (1.5 μm) of red color was obtained on the glass substrate provided with black matrix.

Next, color filter layers of blue and green colors were formed in a similar manner, whereby color filters of these colors were obtained.

What is claimed is:

1. A coating solution for forming a wettability-varied pattern, wherein the coating solution has pH in a neutral region and contains titanium oxide and polysiloxane having a substituent group which is liquid-repellant and directly bonded to each Si atom constituting polysiloxane.

2. The coating solution for forming a wettability-varied pattern according to claim 1, wherein the coating solution further comprises alkylsilicate.

3. The coating solution for forming a wettability-varied pattern according to claim 2, wherein the substituent group which is liquid-repellant is a fluoroalkyl group.

4. The coating solution for forming a wettability-varied pattern according to claim 2, wherein the polysiloxane is a polysiloxane as a hydrolysis condensate or a co-hydrolysis condensate of a silicon compound, which silicon compound includes a silicon compound represented by YₙSiX₍₄₋ₙ₎ (Y represents an alkyl, fluoroalkyl, vinyl, amino, phenyl or epoxy group, X represents an alkoxyl group or a halogen, and n is an integer in a range of 0 to 3).

5. A method of producing a coating solution for forming a wettability-varied pattern, comprising mixing a neutral sol solution of titanium oxide, whose pH is in a neutral range and which contains titanium oxide and alkyl silicate, with a solution of hydrolyzed fluoroalkylsilane, thereby preparing a coating solution for forming a wettability-varied pattern, wherein pH of the solution of hydrolyzed fluoroalkylsilane is adjusted in advance such that pH of the prepared coating solution for forming a wettability-varied pattern is in a range of 5 to 9.

6. A method of producing a pattern-formed body, comprising the processes of:
forming a wettability-variable layer whose wettability at a portion irradiated with energy is modified such that a contact angle with liquid at the portion is decreased, by coating a base material with a coating solution for forming a wettability-varied pattern whose pH is in a neutral region and which contains titanium oxide and polysiloxane having a substituent group which is liquid-repellant directly bonded to each Si atom constituting the polysiloxane and then drying or hardening the coating; and
forming, on the wettability-variable layer, a wettability-varied pattern which is constituted of a lyophilic region and a liquid-repellant region, by irradiating the wettability-variable layer with energy in a pattern-like configuration.

7. The method of producing a pattern-formed body according to claim 6, further comprising the process of preparing a coating solution for forming a wettability-varied pattern, in which process a sol solution of titanium oxide containing the titanium oxide and alkylsilicate is mixed with a solution of hydrolyzed polysiloxane, prior to the wettability-variable layer forming process.

8. The method of producing a pattern-formed body according to claim 6, further comprising the process of filtering the coating solution for forming a wettability-varied pattern, in which process the coating solution for forming a wettability-varied pattern is filtered prior to the wettability-variable layer forming process.

9. The method of producing a pattern-formed body according to claim 6, wherein coating of the coating solution for forming a wettability-varied pattern during the wettability-variable layer forming process is carried out by a method selected from the group consisting of spin coating, slit coating, bead coating, spray coating, dip coating, and combination of slit coating and spin coating.

10. The method of producing a pattern-formed body according to claim 6, wherein the coating solution for forming a wettability-varied pattern is dried or hardened in the wettability-variable layer forming process by drying the coating with a hot plate, an IR heater or an oven.

11. The method of producing a pattern-formed body according to claim 6, wherein irradiation of energy in the wettability-varied pattern forming process is effected by way of a mask.

12. The method of producing a pattern-formed body according to claim 6, wherein a light-shielding portion is formed on the base material so that energy irradiation in the wettability-varied pattern forming process is carried out from the base material side.

13. The method of producing a pattern-formed body according to claim 6, wherein irradiation of energy in the wettability-varied pattern forming process is effected with laser.

14. A method of producing a functional element, comprising the process of forming a functional portion, in which process a functional portion is formed on the wettability-varied pattern of the pattern-formed body produced by the method of producing a pattern-formed body according to claim 6.

15. The method of producing a functional element according to claim 14, wherein the functional portion forming process is carried out by coating or discharge from a nozzle.

16. A color filter, comprising a pixel portion which is the functional portion of the functional element produced by the method of producing a functional element according to claim 14.

17. A microlens, comprising a lens which is the functional portion of the functional element produced by the method of producing a functional element according to claim 14.

18. A conductive pattern, comprising a metal wiring which is the functional portion of the functional element produced by the method of producing a functional element according to claim 14.

19. A base material for biochip, comprising the functional portion of the functional element produced by the method of producing a functional element according to 14, the functional portion being attachable to a biomaterial.

20. An organic electroluminescent (EL) element, comprising an organic EL layer which is the functional portion of the functional element produced by the method of producing a functional element according to claim 14.

21. A coating device using mixture of two types of liquids, the device being used for the method of producing a-pattern-formed body according to claim 6, comprising:
a neutral titanium oxide sol solution accommodating section for accommodating a neutral sol solution of titanium oxide;
a hydrolyzed solution accommodating section for accommodating a solution of hydrolyzed fluoroalkylsilane;
a stirring section connected to the neutral titanium oxide sol solution accommodating section and the hydrolyzed solution accommodating section such that the neutral sol solution of titanium oxide and the solution of hydrolyzed fluoroalkylsilane can be supplied to the stirring section and stirred therein; and
a coating section for coating the base material with a coating solution for forming a wettability-varied pattern, which is prepared by the stirring of the two solutions at the stirring section.
